(19) United States
(12) Patent Application Publication
(10) Pub. No.: US 2002/0173691 A1
(43) Pub. Date: Nov. 21, 2002

(54) Benign method using electromagnetic fields to improve cardiovascular cytoprotection

(76) Inventors: Reba Goodman, Englewood, NJ (US); Hana Lin, Fort Lee, NJ (US); Martin Blank, Tenafly, NJ (US); Hal Skopicki, New York, NY (US)

Correspondence Address: Cooper & Dunham LLP, 1185 Avenue of the Americas, New York, NY, 10036, US

(21) Appl. No.: 09/859,038

(22) Filed: May 15, 2001

(51) Int. Cl. A61N 2/00

(52) U.S. Cl. 600/9

(57) ABSTRACT

A unique approach to clinical application of cytoprotection is offered by electromagnetic (EM) field induction of stress proteins. Electromagnetic fields are non-invasive and easily applied, compared to the current hyperthermia protocols. Fertilized dipteran eggs and cultured rodent cardiomyocytes (H9c2 cells) were used as first-level models to test electromagnetic fields for their ability to induce increased hsp70 levels for effective cytoprotection. Eggs preconditioned with an 8 μT 60 Hz EM field for 30 minutes had a 114% increase in hsp70 levels, and an average 82% increase in survival following a lethal temperature of 36.5° C. Thermal pre-conditioning at 32° C. was not nearly as effective in dipteran eggs, inducing only a 44% increase in survival. Preconditioning of cultured murine cardiomyocytes (H9c2 cells) with an 8 μT 60 Hz field induced a 77% average increase in hsp70 levels.

### BACKGROUND OF THE INVENTION

The present invention relates to a benign method to improve cardiovascular cytoprotection using electromagnetic fields.

Throughout this application, various publications are referenced to by arabic numerals within parentheses. Disclosures of these publications in their entireties are hereby incorporated by reference into this application to more fully describe the state of the art to which this invention pertains. Full bibliographic citations for these references may be found at the end of this application, preceding the claims.

There is increasing evidence that stress proteins/chaperones play crucial and protective roles in a variety of cellular processes, including repair or degradation of damaged proteins, as well as contributing to cytoprotection (31). As the role of cytoprotective chaperones in cardioprotection in ischemic heart disease has become more widely understood, their induction prior to surgery has become more prevalent. To induce these proteins, clinical procedures have utilized whole body hyperthermia in an effort to protect the mycardium during reperfusion ischemic stress following stroke and heart attack (2).

However, this approach has a number of drawbacks, in that such approach is resisted by the body, is cumbersome, is difficult to administer with precision, and is decidedly uncomfortable for the patient.

### SUMMARY OF THE INVENTION

The present invention provides a benign alternative to hyperthermia. Electromagnetic field exposure profiles are provided; electromagnetic fields can induce increased cytoprotective protein (hsp70) levels through carefully delineated exposures. The present invention has been shown to have positive results in an in vivo system (e.g., fertilized dipteran eggs).

The present invention may be used for treatment prior to cardiac bypass surgery. Furthermore, the present invention may be used to protect the myocardium before and during reperfusion ischemic stress to prevent heart attack and stroke. The present invention may also be applied immediately upon onset of a heart attack.

Using the procedure of the present invention is less cumbersome than hyperthermia while also improving the comfort level of patients. Furthermore, the procedure of the present invention is safe, non-invasive and has longer-lasting effects. Moreover, application of electromagnetic fields is also considerably easier and more precise for the clinician.

Therefore, the present invention provides a precise, simple, gentle, and patient-friendly procedure that is easily applied. Unlike hyperthermia, cytoprotective protein (hsp70) levels can be restimulated with different electromagnetic field strengths for extended effects.

In summary, the present invention in one embodiment provides a non-invasive method for cardiovascular cytoprotection, comprising the step of applying an electromagnetic field to a subject to induce increased levels of stress protein hsp70 in the subject.

The present invention in another embodiment provides a non-invasive method for cardiovascular cytoprotection, comprising the steps of applying an electromagnetic field to a subject to induce increased levels of stress protein hsp70 in the subject, and restimulating the levels of stress protein hsp70 after the applying step by applying a plurality of different electromagnetic field strengths to the subject.

### BRIEF DESCRIPTION OF THE FIGURES

FIG. 1 illustrates photographs of larvae from preconditioned eggs, compared to eggs not preconditioned;

FIG. 2 illustrates the percent survival of fertilized dipteran eggs under various exposure conditions;

FIG. 3 illustrates hsp70 levels in dipteran eggs with and without electromagnetic field preconditioning;

FIG. 4 illustrates hsp70 levels in cardiomyocytes exposed to electromagnetic fields and heat shock; and

FIG. 5 illustrates hsp70 levels in cardiomyocytes exposed to electromagnetic fields and/or heat shock.

### DETAILED DESCRIPTION OF THE INVENTION

The present invention in one embodiment provides a noninvasive method for cardiovascular cytoprotection in an in vivo system, comprising applying an electromagnetic field to a subject to induce increased levels of stress protein hsp70 in the subject.

The electromagnetic field may be applied at a low frequency and at a low field strength. The electromagnetic field may be applied at a field strength of 8 μT and at a frequency of 60 Hz. The electromagnetic field may be applied for an applying time of approximately 30 minutes. The electromagnetic field may be applied at a field strength of 80μT.

Further stress proteins may be induced, including hsp27 and hsp90. The electromagnetic field may be applied in a series of exposures. The method may be used prior to cardiac bypass surgery. The method may be used for myocardium protection during reperfusion ischemic stress to prevent a heart attack.

The levels of stress protein hsp70 may be restimulated following the applying time using a plurality of different electromagnetic field strengths.

The levels of stress protein hsp70 may be restimulated using a plurality of different electromagnetic field strengths for approximately 30 minutes at any time during an approximate three-hour period following the applying time.

The present invention in another embodiment provides a noninvasive method for cardiovascular cytoprotection in an in vivo system, comprising the steps of applying an electromagnetic field to a subject to induce increased levels of stress protein hsp70 in the subject, and restimulating the levels of stress protein hsp70 after the applying step by applying a plurality of different electromagnetic field strengths to the subject.

The applying step may last for an applying time of approximately 30 minutes. The restimulating step may be performed at any time during an approximate three-hour period following the applying time and lasts for approximately 30 minutes.

The electromagnetic field may be applied at a low frequency and at a low field strength. Further stress proteins may be induced, including hsp27 and hsp90. The electromagnetic field may be applied during the applying step at a field strength of 8 μT and at a frequency of 60 Hz. The electromagnetic field may be applied during the applying step at a field strength of 80 μT. The electromagnetic field may be applied during the applying step in a series of exposures.

A benign, non-invasive method of inducing increased levels of the stress protein hsp70 can be accomplished through the application of short exposures to low-energy, low-frequency electromagnetic fields (13).

There are a number of advantages to electromagnetic fields over hyperthermia.

Firstly, low frequency electromagnetic fields penetrate all cells, essentially without attenuation, as opposed to high temperatures (4).

Secondly, electromagnetic fields and heat induce stress proteins at very different energy input levels; the energy density required for a 0.8 μT EM field to induce increased hsp70 levels is 14 orders of magnitude lower than the 5.5° C. temperature rise to elicit a comparable effect (13).

Thirdly, cells respond rapidly to electromagnetic fields; exposure for only 10 seconds elicits a measurable response (21).

Fourthly, significant levels of hsp70 are induced well within 30 minutes from onset of electromagnetic field exposure (3) (12), and the levels remain elevated for more than three hours (15).

Furthermore, unlike thermally induced (‘heat shock’) stress proteins, electromagnetic field-induced protein levels can be restimulated with different field strengths (higher or lower) to even greater hsp70 levels (13) (15).

Moreover, electromagnetic field-induced protection will occur even if the field is applied immediately after the start of hypoxia, a condition that mimics treatment following onset of heart attack (10).

The induction of these protective proteins by electromagnetic fields provides significant advantages in ease and simplicity of application for both the patient and the clinician. At the same time, it offers equivalent cardioprotection to hyperthermia. Because the ideal preconditioning agent would be non-invasive and easily administered in precise doses, the potential of stress protein induction by electromagnetic fields is medically attractive and promising. Electromagnetic fields have been used since the 1970s for healing bone non-unions and wound healing, and for the reduction of inflammation (1).

Experimental Details

To develop a protocol for electromagnetic field induction of hsp70 for cytoprotection, fertilized dipteran eggs were selected as a first-level model. The rationale for such selection was that the conditions for optimal cytoprotection could ultimately be used to provide a protocol for experiments with a mouse infarction model. In some experiments, electromagnetic preconditioning induction of hsp70 levels in fertilized eggs (an in vivo model) was compared to electromagnetic preconditioning induction of hsp70 in cultured rat cardiomyocytes (an in vitro model).

Materials and Methods

Growth and Maintenance of Sciara coprophila

Conditions for growth and maintenance of Sciara coprophila have been extensively described (7) (11) (32). For the purposes of this study, Sciara provided several distinct advantages over the more well-known dipteran model, Drosophila. Each Sciara female deposits approximately 75 eggs simultaneously four days after mating. In Drosophila, smaller numbers of eggs are deposited, and over a wider time frame. With appropriate matings it can be predetermined whether deposited eggs hatch into all female or all male adult flies using the wing marker “curly”; that is, phenotypic evidence of the presence of a gene that determines that eggs deposited by female flies carrying this wing marker will hatch into all female adult flies.

Rodent Cardiomyocytes

Cardiomyocytes (ATCC; H9c2) were grown in DMEM and maintained as indicated by American Type Culture Collection. Cells were treated with electromagnetic fields or heat at sub-confluency, 1×10⁻⁶ cells/ml, in 15 mls in Petri dishes (100 mm).

Establishing EM and Temperature Pretreatment Conditions

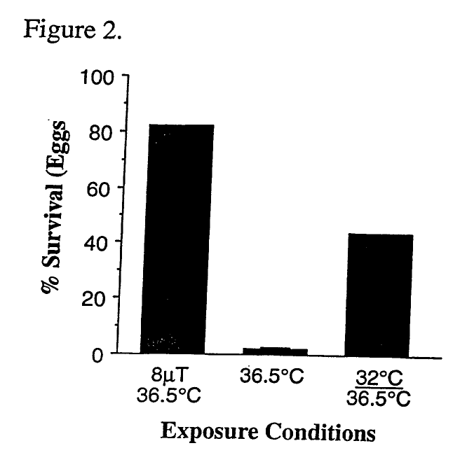

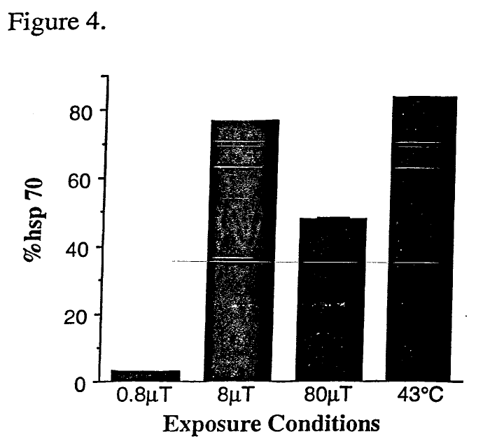

To determine optimum field strength for survival and hsp70 induction, dipteran eggs (and cardiomyocytes) were exposed to field strengths of 0.8, 8, and 80 μT at a frequency of 60 Hz. 8 μT was selected as a preconditioning field strength based on percent survival following a lethal temperature as well as the level of hsp70 induced (see FIGS. 2 and 4, described in further detail below).

FIG. 2 illustrates the percent survival of fertilized dipteran eggs under various exposure conditions. First, the percent survival is shown of eggs preconditioned with an 8 μT EM field (30 minutes) followed by exposure to a lethal temperature (36.5° C. for 60 min) (82%±0.16). Second, the percent survival is shown of eggs exposed 60 minutes to a lethal temperature (36.5° C.) (2%±0.56). Third, the percent survival is shown of eggs thermally preconditioned (32° C.) followed by exposure to a lethal temperature (36.5° C.) (44%±0.01). Data are the averages from twelve experiments (±standard error of the mean).

FIG. 4 illustrates hsp70 levels in cardiomyocytes exposed to electromagnetic fields and heat shock under various conditions. FIG. 4 shows the following:

hsp70 in cells exposed to 0.8 μT=2.98%±0.08

hsp70 in cells exposed to 8μT=77%±0.12

hsp70 in cells exposed to 80 μT=48%±0.31

hsp70 in cells exposed to 43° C.=83%±0.31

The data is averaged from eight Western blots.

To determine lethal and sub-lethal temperatures, eggs were exposed to temperatures from 30° C. to 39° C. 36.5° C. was the lethal temperature and 32° C. the sublethal temperature. Sublethality was based on survival of eggs hatching into larvae after 60 minutes of exposure to a lethal temperature of 36.5° C. Heat shock for cardiomyocytes was 43° C.

Temperature Control

Temperature was monitored with a Physitemp (BAT-12) thermocouple probe (PhysiTemp, Hackensack, New Jersey), (sensitive to ±0.1° C.) to ensure that no heating resulted from the Helmholtz coils. The thermocouple probe was attached to the Helmholtz coils throughout all electromagnetic field exposures.

Heat Shock

Petri dishes containing the eggs or cardiomyocytes were wrapped in Parafilm, placed in mu metal containers and immersed in a water bath. Mu metal (described in further detail below) shields against stray electromagnetic fields, in this case the fields generated by the heating unit in the water bath.

Conditions for Collecting Dipteran Eggs

Four females (curly winged) and four males were mated in each 100 mm Petri dish containing a 1 cm layer of 4% agar. Eggs were counted four days after mating and the number of eggs was recorded. An accurate egg count was obtained using a 1.5 cm×1.5 cm grid on the bottom of each Petri dish.

Protocol for EM Field and Thermal Conditions for Eggs

Petri dishes containing eggs were grouped as follows:

Group 1: Eggs preconditioned with 8 μT 60 Hz EM fields for 30 minutes at 20° C., followed by recovery for an additional 30 minutes. Eggs were then exposed to a temperature of 36.5° C. for 60 min and returned to the 20° C. incubator.

Group 2: Eggs with no EM field preconditioning and exposed for 60 minutes to a lethal temperature (36.5° C.). These eggs served as controls for Group 1.

Group 3: Eggs preconditioned with 32° C. heat for 30 minutes followed by a 30 minute recovery at room temperature. The eggs were then exposed to a lethal temperature of 36.5° C. for 60 minutes and returned to 20° C. incubator.

Group 4: Eggs with no heat preconditioning and exposed to a lethal temperature (36.5° C.). These eggs served as controls for Group 3.

Protocol for EM Field and Thermal Conditions for Cardiomycocytes

Petri dishes containing cells (1×10⁶ cells/ml in 15 mls) were grouped as follows.

Group 1: preconditioned 8 μT EM fields for 30 min at 37° C., followed by recovery for an additional 30 minutes (and protein extracted)

Group 2: 8 μT EM preconditioned cells exposed to 43° C. for 60 minutes (and protein extracted)

Group 3: no preconditioning (and protein extracted).

Group 4: no preconditioning and exposed to a 43° C. lethal temperature for 30 minutes (protein extracted).

Determination of Percent Survival for Dipteran Eggs

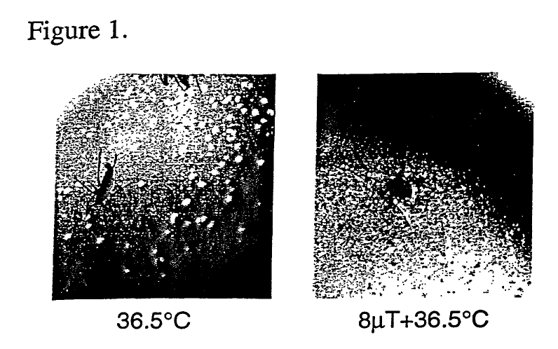

Seven days following experimental or control treatment, Petri dishes were examined and moving larvae were counted and recorded. FIG. 1 shows photographs of larvae from preconditioned eggs, compared to eggs not preconditioned. The left side shows eggs (white dots) that were subjected to lethal heat shock at 36.5° C. without prior conditioning by electromagnetic fields, and that did not hatch into larvae. The right side shows moving larvae (small black dots) that hatched from pre-conditioned eggs (8 μT EM field) exposed to lethal heat shock at 36.5° C. The number of surviving larvae was compared to the initial number of eggs in each dish, and survival percentages were calculated.

EM Field Exposures

Two fully functional exposure units provided simultaneous sham and experimental exposures. Exposures used Helmholtz coils (Electric Research and Management, Pittsburgh, Pa.) that consisted of 19-gauge wire bundles wound 164 times around a square form 13 cm long and 14 cm wide with 8 cm spacing. The coils were energized by a function generator (11 MHz Wavetek Stabilized Function Generator; model 21). A digital multimeter was used to measure the field intensity and verify the systems operation (Fluke 87 digital multimeter). Field parameters were monitored with a Hitachi V-1065 100 MHz oscilloscope and a calibrated inductive search coil (25×; Electro-Biology Inc., Parsippany, N.J.). Detailed description of the exposure system, including background magnetic fields in the incubator, harmonic distortion, DC magnetic fields and mean static magnetic fields in the incubator, both vertical and horizontal components, can be found in Jin et al, 1997.

Cells were placed on a plexiglas stand in a horizontal orientation; i.e., the entire area of the dish was exposed to the field. The bottom of the dish was 2 cm below the axis level. The height from dish bottom to top surface of liquid or the agar was approximately 1.1 cm. The height of the liquid or agar was 0.6 cm. The calculated electric field was ˜11 μV/m for an 8 μT exposure.

Mu Metal Shielding

Helmholtz coils were enclosed within Mu metal containers to prevent Petri dishes containing cardiomyocytes and eggs from stray fields during all electromagnetic field exposures. Both active (experimental) and sham-exposed coils (controls) were enclosed in a 30 cm high, 15 cm diameter cylindrical mu metal container (0.040″ thickness) (Amuneal Corp. Phlidelphia, Pa.). The 60 Hz shielding factor is (Min.) 90.1 (39.08 dB). Sham-exposed controls and experimental exposures are performed simultaneously in identical mu metal containers.

Protein Lysates

Lysates were prepared from H92c cardiomyocyte cells as previously described (Lin et al 1997; modification of Mosser et al 1988). Extraction of protein from eggs was accomplished by treating the eggs alternately with dry ice (30 mins) and room temperature water (60 mins) for 2 hrs, together with glass beads. Protein concentrations were determined with a Bio-Rad protein assay kit (Bio-Rad Laboratories).

Western Blot

Protein lysates from dipteran eggs and cardiomyocytes were analyzed for hsp70 levels using the ECL detection system (Amersham). The intensity of the signal was determined with a PhosphorImager 400A (Molecular Dynamics) and was quantified using ImageQuant software.

Antibodies

The human anti-hsp70 was supplied by StresGen; the Drosophila anti hsp70.1 was provided by Dr. Susan Lindquist, University of Chicago.

Statistical Analyses

A sufficient number of experiments were performed to assure statistical significance. Samples from each experiment were tested three times and experiments were repeated a minimum of six times. The data were entered into Excel for analysis and the results were examined with a two-tailed t-test. Statistical significance is determined by multifactor analysis of a variance program (INSTAT). The data is expressed as the ratio of the experimental to the control (E/C)±standard error of the mean.

Results

Electromagnetic field preconditioning induces higher survival than thermal preconditioning. To determine percent survival, the number of eggs that hatched into moving feeding larvae seven days after exposure to 36.5° C. was divided by the initial number of preconditioned eggs (see FIG. 1). FIG. 2 shows that Sciara eggs preconditioned with an 8 μT EM field for 30 minutes had an 82% increase in survival to a temperature of 36.5° C., compared to a 2.5% survival of eggs that were not preconditioned. Eggs thermally pre-conditioned at 32° C. had a 44% increase in survival to 36.5° C.

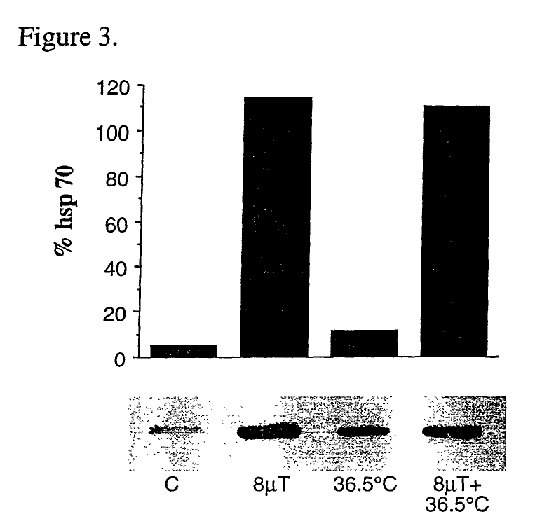

Hsp70 levels induced by EM fields in dipteran eggs and rodent cardiomyocytes. FIG. 3 shows an example of hsp70 levels in dipteran eggs with and without electromagnetic field preconditioning, as determined by Western blot. Eggs pretreated or preconditioned with an 8 μT EM field showed a 114% increase in hsp70 levels (lane 2). Eggs pretreated with an 8 μT EM field and exposed to 36.5° C. had a similar increase (110%) in hsp70 (lane 4). There was a 12% increase in hsp70 in unpreconditioned eggs heat shocked at 36.5° C. (lane 3). Hsp70 levels in eggs with no pre- or post-treatment (controls) were lower than 5% (lane 1).

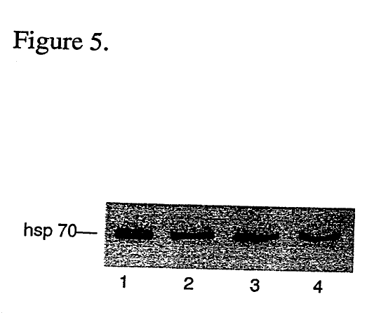

FIG. 4 shows the average levels of hsp70 in cultured cardiomyocytes exposed to three different electromagnetic field strengths. Exposure to an 8 μT EM field increased hsp70 levels 77% (±0.12%), whereas exposure to an 80 μT EM field increased hsp70 levels only 48% (±0.31%). Exposure to a 0.8 μT field induced virtually no increase in hsp70—only 2.9%. Cells heat shocked at 43° C. showed an 83% (±0.31%) increase in hsp70. FIG. 5 shows an example of a Western blot from these experiments.

More specifically, FIG. 5 shows hsp70 levels in cardiomyocytes exposed to electromagnetic fields and/or heat shock. Lane 1 shows protein from cells exposed to heat shock at 43° C. Lane 2 shows protein from cells exposed cells to an 8 μT EM field followed by heat shock. Lane 3 shows protein from cells exposed to an 8 μT EM field. Lane 4 shows protein from sham-exposed cells.

Discussion

Based on the results from the experiments described here, it is proposed that the clinical benefits due to induced increases in hsp70 levels can be achieved just as effectively by exposure to electromagnetic fields as by hyperthermia. Although our studies have focused on the major stress protein hsp70, electromagnetic fields induce increased levels of hsp27 and 90 as well (15). As in “heat shock,” electromagnetic fields induce activation of the HSP70 gene expression through trimerization of heat shock factor 1 (HSF1) and HSF1 binding to a heat shock element (HSE) in the HSP70 promoter (18) (19). Similar to “heat shock,” there is an electromagnetic field domain in the promoter region of the HSP70 gene, with a well-defined response element that differs from the “heat shock” domain. Deletion of the electromagnetic field response element results in a 100% reduction in the electromagnetic field induction of hsp70 (20).

Of particular interest in terms of clinical application is our observation that the increase in hsp70 protein levels induced by a 30 minute electromagnetic field exposure persists for more than three hours, and that a 30 minute restimulation with a different (higher or lower) electromagnetic field strength at any time during this three hour period induced even higher levels of hsp70 (15).

Furthermore, there are some detrimental side effects associated with “heat shock” and its clinical application, hyperthermia, that are avoided by the use of electromagnetic fields. Firstly, the messenger RNAs for basal (“nonessential”) cellular proteins, normally inhibited by “heat shock,” are not affected by EM field-induced stress. Secondly, although “heat shock” is effective with cells and isolated tissues, hyperthermia has serious limitations when applied to human patients due to normal resistance by the body's thermoregulatory mechanisms.

Cardiovascular Cytoprotection with Heat-Induced hsp70

The induction of hsp70 for clinical application is a rapidly growing area of experimental medical research. Transgenic mice overexpressing hsp70 have been reported to decrease infarct size (26) and extra gene copies have been shown to support cell integrity, viability and function in tissues such as the heart (2) (17) (27) (28) (29) (30 (31).

Most damage incurred by ischemic events is suffered at reperfusion, when blood flow and oxygen supplies resume (29). It is believed that generation of high concentrations of oxygen radicals during this period are responsible for extensive cellular damage. In many cases the endogenous synthesis of stress proteins, induced primarily at reperfusion, is too late to prevent significant damage. In an embryonic chick heart model, electromagnetic field-induced protection is also conferred when the fields are activated after hypoxic conditions have developed, but prior to reperfusion (10).

Pretreatment with heat stress has been successfully used with whole animals to improve recovery from ischemia (8) (9), and increase resistance of donor hearts to the rigors of cryopreservation and subsequent warming for transplantation (14). A 100% induction of hsp70 strengthens heart muscle cell resistance to oxidation, ischemia and hypoxia (5) (16) (23) (24). During transient myocardial ischemia in vivo, concentrations of endogenous hsp70 have been shown to increase, doubling one hour after coronary artery occlusion (22). When increases in stress proteins reach at least four to five times baseline (which may not occur for up to 24 hours after the ischemic stress), improved ischemic tolerance (e.g., recovery of cardiac output and left ventricular pressure) in myocardial tissue is noted (6). Unfortunately, this is outside the “golden window of opportunity” to protect the mycardium, which is at its greatest risk the first six hours after artery occlusion.

Central to the reduction of risk posed to the heart by all major surgery, and for the treatment of myocardial infarction and heart failure, is the protection of heart muscles against ischemia and hypoxia. Based on data from the model reported here, as well as the increased hsp70 following repeated short electromagnetic field exposures (15), we believe that the induction of hsp70 by electromagnetic fields can be developed as an effective and efficient therapeutic intervention. Investigation with mammalian models is the next step in the development of this technique.

Although embodiments of the invention have been described herein, numerous variations and modifications will occur to those skilled in the art without departing from the scope of the invention. The invention is not limited to the embodiments disclosed, and is defined only by way of the following claims.

### REFERENCES

1. C. A. L. Bassett. Bioelectromagnetics in the Service of Medicine. Adv Chem 250: 261-276 (1995).

2. I. J. Benjamin, D. R. McMillan. Molecular Chaperones in Cardiovascular Biology and Disease. Circ Res, 83: 117-132 (1998).

3. M. Blank, O. Khorkova, R. Goodman. Changes in Polypeptide Distribution Stimulated by Different Levels of Electromagnetic and Thermal Stress. Bioelectrochem Bioenerg 33, 109-114 (1994).

4. M. Blank and R. Goodman. Electromagnetic Fields May Act Directly on DNA. J Cellular Biochem 75: 369-374 (1999).

5. K. Y. Chong, C. C. Lai, S. Lille, C. Chang, C. Su Stable. Overexpression of the Constitutive Form of Heat Shock Protein 70 Confers Oxidative Protection. J Mol Cell Cardiol 30, 599-608 (1998).

6. R. N. Cornelussen, A. V. Garnier, G. J. van der Vusse, R. S. Reneman, L. H. E. H. Snoeckx. Biphasic Effect of Heat Stress Pretreatment on Ischemic Tolerance of Isolated Rat Heart. J Mol Cell Cardiol 30:365-72 (1998).

7. H. V. Crouse. X Heterochromatin Subdivision and Cytogenetic Analysis in Sciara Coprophila I. Centromere localization Chromosoma 53: 39-55 (1977).

8. R. W. Currie, R. Tanguay, J. G. Klingma. Heat-shock Response and Limitation of Tissue Necrosis During Occlusion/Reperfusion in Rabbit Hearts. Circulation 87:863-71 (1993).

9. T. J. Donnelly, R. E. Sievers, F. L. J. Vissern, W. J. Welch, C. L. Wolfe. Heat Shock Protein Induction in Rat Heart. A role for improved myocardial salvage after ischemia and reperfusion? Circulation 85: 769-778 (1992).

10. A. L. DiCarlo, J. M. Farrell, T. A. Litovitz. Myocardial Protection Conferred by Electromagnetic Fields. Circulation: 99: 813-816 (1998).

11. N. Gabrusewycz-Garcia. Cytological and Autoradiographic Studies in Sciara Coprophila Salivary Gland Chromosomes. Chromosoma 15: 312-344 (1964).

12. R. Goodman and A. Henderson. Exposure of Salivary Gland Cells to Low-Frequency Electromagnetic Fields Alters Polypeptide Synthesis. Proc Natl Acad Sci (USA) 85, 3928-3932 (1988).

13. R. Goodman and M. Blank. Magnetic Field Stress Induces Expression of Hsp70. Cell Stress & Chaperones 3: 79-88 (1998).

14. A. Gowda, C. Yang, G. K. Asimakis, S. Rastegar, M. Motamedi. Heat Shock Improves Recovery and Provides Protection Against Global Ischemia After Hypothermic Storage. Ann Thorac Surg 66:1991-7 (1998).

15. L. Han, H. Lin, M. Jin, M. Blank, R. Goodman. Application of Magnetic Field-Induced Heat Shock Protein 70 for Presurgical Cytoprotection. J Cell Biochem 71: 577-583 (1998).

16. R. J. Heads, D. S. Latchman, D. M. Yellon. Stable High Level of Expression of a Transfected Hsp70 Gene Protects a Heart-Derived Muscle Cell Line Against Thermal Stress. J Mol Cardiol 26, 695-699 (1994).

17. K. Iwaki, S-H. Chi, W. H. Dillman, R. Mestril. Abnormal Proteins Serve as Eukaryotic Stress Signals and Trigger Activation of Heat Shock Genes. Science (Washington, D.C.) 232: 252-254 (1993).

18. H. Lin, M. Opler, M. Head, M. Blank, R. Goodman. Electromagnetic Field Exposure Induces Rapid, Transitory Heat Shock Factor Activation in Human Cells. J Cell Biochem 66: 482-488 R (1997).

19. H. Lin, M. Head, M. Blank, L. Han, M. Jin, R. Goodman. Myc-Mediated Transactivation of HSP70 Expression Following Exposure to Magnetic Fields. J Cellular Biochem 69:181-188 (1998).

20. H. Lin, M. Blank, R. Goodman. Magnetic Field-Responsive Domain in the Human HSP70 Promoter. J Cell Biochem 75: 170-176 (1999).

21. T. A. Litovitz, D. Krause, J. M. Mullins. Effects of Coherence Time of the Applied Magnetic Field on Ornithine Decarboxylase Activity. Biochem Biophys Res Commun 178, 862-865 (1991).

22. R. Loncar, C. W. Flesche, A. Deussen. Regional Myocardial Heat-Shock Protein (HSP70) Concentrations Under Different Blood Flow Conditions. Pflugers Arch 437:98-103 (1998).

23. R. Mestril, F. J. Giordano, A. G. Conde, W. H. Dillmann. Adenovirus-Mediated Gene Transfer of a Heat Shock Protein 70 (hsp) Protects Against Stimulated Ischemia. J Mol Cell Cardiol. 26: 2351-8 (1996).

24. R. Mestril, S-H. Chi, M. R. Sayen, K. O'Reilly, W. H. Dillman Expression of Inducible Stress Protein in Rat Heart Myogenic Cells Confers Protection Against Simulated Ischaemia-Induced Injury. J Clin Invest 9, 759-767 (1994).

25. D. D. Mosser, N. G. Theodrakis, R I. Morimoto. Coordinate Changes in Heat Shock Element-Binding Activity and HSP70 Gene Transcription Rates in Human Cells. Mol Cell Biol 8, 4736-4744 (1988).

26. J. C. L. Plumier, B. Ross, R. W. Currie, C. E. Angelidis, H. Kazlaris, G. Kollias, G. N. Pagoulatous. Transgenic Mice Expressing the Human Heat Shock Protein 70 Have Improved Post-Ischemic Myocardial Recovery. J Clin Invest. 95:1854-60 (1995).

27. J-C L. Plumier, R. W. Currie, Heat Shock-Induced Myocardial Protection Against Ischemic Injury: A Role for Hsp70? Cell Stress & Chaperones 1, 13-17 (1996).

28. R. Udelsman, M. J. Blake, C. A. Stagg, D-G. Li, D. Putney, N. J. Holbrook. Vascular Heat Shock Protein Expression in Response to Stress. J Clin Invest 91, 465-473 (1993).

29. D. M. Yellon and D. S. Latchman. Stress Proteins and Myocardial Protection. J Mol Cardiol 24: 113-124 (1992).

30. R. S. Williams, J. A. Thomas, M. Fina, Z. German, I. J. Benjamin Human Heat Shock Protein 70 Protects Murine Cells From Injury During Metabolic Stress. J Clin Invest 92, 503-508 (1993).

31. W. J. Welch and C. R. Brown. Influence of Molecular and Chemical Chaperones on Protein Folding. Cell Stress & Chaperones 1: 109-115 (1996).

32. E. Zegarelli-Schmidt and R. Goodman. The Diptera As a Model System In Cell and Molecular Biology. In: “International Rev of Cytol,” Vol. 71.Bourne J and Danielli J (eds) Academic Press, New York, pp 245-363 (1981).

We claim:

1. A non-invasive method for cardiovascular cytoprotection in an in vivo system, comprising applying an electromagnetic field to a subject under conditions sufficient to induce increased levels of stress protein hsp70 in the subject.

2. The non-invasive method for cardiovascular cytoprotection in an in vivo system as set forth in claim 1, further comprising the step of applying the electromagnetic field at a low frequency and at a low field strength.

3. The non-invasive method for cardiovascular cytoprotection in an in vivo system as set forth in claim 1, further comprising the step of applying the electromagnetic field at a field strength of 8 μT and at a frequency of 60 Hz.

4. The non-invasive method for cardiovascular cytoprotection in an in vivo system as set forth in claim 1, further comprising the step of applying the electromagnetic field for an applying time of approximately 30 minutes.

5. The non-invasive method for cardiovascular cytoprotection in an in vivo system as set forth in claim 1, further comprising the step of applying the electromagnetic field at a field strength of 80 μT.

6. The non-invasive method for cardiovascular cytoprotection in an in vivo system as set forth in claim 1, wherein the step of applying is under conditions sufficient to induce further stress proteins including hsp27 and hsp90.

7. The non-invasive method for cardiovascular cytoprotection in an in vivo system as set forth in claim 1, wherein the electromagnetic field is applied in a series of exposures.

8. The non-invasive method for cardiovascular cytoprotection in an in vivo system as set forth in claim 1, wherein the method is used prior to cardiac bypass surgery.

9. The non-invasive method for cardiovascular cytoprotection in an in vivo system as set forth in claim 1, wherein the method is used for myocardium protection during reperfusion ischemic stress following a heart attack.

10. The non-invasive method for cardiovascular cytoprotection in an in vivo system as set forth in claim 4, further comprising the step of:
restimulating the levels of stress protein hsp70 following the applying time using a plurality of different electromagnetic field strengths.

11. The non-invasive method for cardiovascular cytoprotection in an in vivo system as set forth in claim 4, further comprising the step of:
restimulating the levels of stress protein hsp70 using a plurality of different electromagnetic field strengths for approximately 30 minutes at any time during an approximate three-hour period following the applying time.

12. A non-invasive method for cardiovascular cytoprotection in an in vivo system, comprising the steps of:
applying an electromagnetic field to a subject under conditions sufficient to induce increased levels of stress protein hsp70 in the subject; and
restimulating the levels of stress protein hsp70 after the applying step by applying a plurality of different electromagnetic field strengths to the subject.

13. The non-invasive method for cardiovascular cytoprotection in an in vivo system as set forth in claim 12, wherein the applying step lasts for an applying time of approximately 30 minutes.

14. The non-invasive method for cardiovascular cytoprotection in an in vivo system as set forth in claim 13, wherein the restimulating step is performed at any time during an approximate three-hour period following the applying time and lasts for approximately 30 minutes.

15. The non-invasive method for cardiovascular cytoprotection in an in vivo system as set forth in claim 12, further comprising the step of applying the electromagnetic field at a low frequency and at a low field strength.

16. The non-invasive method for cardiovascular cytoprotection in an in vivo system as set forth in claim 12, wherein the step of applying is under conditions sufficient to induce further stress proteins, including hsp27 and hsp90.

17. The non-invasive method for cardiovascular cytoprotection in an in vivo system as set forth in claim 12, wherein the electromagnetic field is applied during the applying step at a field strength of 8 μT and at a frequency of 60 Hz.

18. The non-invasive method for cardiovascular cytoprotection in an in vivo system as set forth in claim 12, wherein the electromagnetic field is applied during the applying step at a field strength of 80 μT.

19. The non-invasive method for cardiovascular cytoprotection in an in vivo system as set forth in claim 12, wherein the electromagnetic field is applied during the applying step in a series of exposures.
